(19) United States
(12) Patent Application Publication
(10) Pub. No.: US 2016/0129230 A1
(43) Pub. Date: May 12, 2016

(54) METHOD OF TREATMENT OF PREMALIGNANT AND MALIGNANT SKIN LESIONS WITH CYTOTOXIC AGENTS

(71) Applicant: ELORAC, LTD, VERNON HILLS, IL (US)

(72) Inventor: Joel E. Bernstein, Deerfield, IL (US)

(21) Appl. No.: 14/988,482

(22) Filed: Jan. 5, 2016

Related U.S. Application Data
(63) Continuation of application No. 12/855,936, filed on Aug. 13, 2010, now abandoned.

(51) Int. Cl. A61M 35/00; A61K 31/365; B65B 3/04; A61M 39/22; A61F 13/40; A61K 31/513; A61K 31/4745

(52) U.S. Cl. CPC A61M 35/003; A61K 31/513; A61K 31/365; A61K 31/4745; A61M 39/22; A61M 35/006; B65B 3/04; A61M 2207/00; A61M 2209/06

(57) ABSTRACT

A method of packaging and applying toxic anti-neoplastic pharmaceutical agents for the prevention and treatment of premalignant and malignant lesions of the skin is described. The packaging of liquid formulations containing these toxic anti-neoplastic agents in plastic or glass bottles with control-flow applicators and caps enables patients to apply such formulations to affected areas of skin without having to have physical contact on the part of their hands with the medicaments.

### CROSS REFERENCE TO RELATED APPLICATION

This application is a continuation of U.S. patent application Ser. No. 12/855,936 filed on Aug. 13, 2010. The disclosure set forth in the referenced application is incorporated herein by reference in its entireties.

### BACKGROUND

Pre-malignant and malignant lesions of the skin have been increasing steadily in incidence, in part due to environmental pollution and increased exposure to ultraviolet light, and in part due to other factors which have, as yet, not been fully elucidated. The principal cutaneous neoplasms which have public health significance are actinic or solar keratoses (pre-malignant skin lesions), basal cell carcinomas, squamous cell carcinomas, condyloma acuminatum, cutaneous t-cell lymphoma (i.e. mycosis fungoides), and malignant melanomas. Of these lesions, solar keratoses, basal cell carcinomas, and condyloma acumination are by far the most common. These are also the easiest to treat and generally have a good prognosis. Squamous cell carcinomas of the skin, cutaneous T-cell lymphomas, and malignant melanomas are less common, but carry a much poorer prognosis.

Treatment approaches to solar keratosis and basal cell carcinomas have generally relied on cryosurgery, electrocautery and curettage, minor surgical procedures (including “Mohs” surgery) or the use of topical 5-fluorouracil (5-FU), a potent cytotoxic agent. Treatment of condyloma acuminatum usually relies on topical application of podophyllotoxin (podofilox), imiquimod or 5-FU. Treatment of squamous cell carcinomas and malignant melanomas may involve more major surgical procedures and systemic chemotherapy.

While commonly used for treatment of solar keratoses, basal cell carcinomas and condyloma acuminatum, the anti-neoplastic and/or immunosuppressant agents 5-FU, podophyllotoxin, and imiquimod are quite toxic to normal skin. Consequently, the labeling of all marketed products containing these active ingredients contains express instructions/warnings to apply the products with plastic gloves and/or wash hands thoroughly after applying.

### SUMMARY OF THE DISCLOSURE

A method of packaging and applying toxic anti-neoplastic pharmaceutical agents for the prevention and treatment of premalignant and malignant lesions of the skin is described. The packaging of liquid formulations containing these toxic anti-neoplastic agents in bottles with control-flow applicators and caps enables patients to apply such formulations to affected areas of skin without having to have physical contact on the part of their hands with the medicaments.

By packaging liquid formulations, including but not limited to solutions, lotions, suspensions, creams and gels, in bottles with control-flow applicators and caps such as those applicators and caps produced by the Dab-O-Matic Corporation, formulations containing these toxic active ingredients can be applied to skin areas affected by solar keratoses, basal cell carcinomas and condyloma acumination without the patient having any hand contact with these formulations. In this manner, such formulations can be applied to skin lesions without the necessity for the patient wearing gloves or thoroughly washing their hands following application. This new method of application also protects the skin of the hands from the severe irritant properties of these toxic active ingredients.

### BRIEF DESCRIPTION OF THE DRAWINGS

FIG. 1A is a photograph of a suitable bottle for practice of the invention described herein;

FIG. 1B is a diagramatic representation of the control-flow applicator and caps.

### DETAILED DESCRIPTION OF THE DISCLOSURE

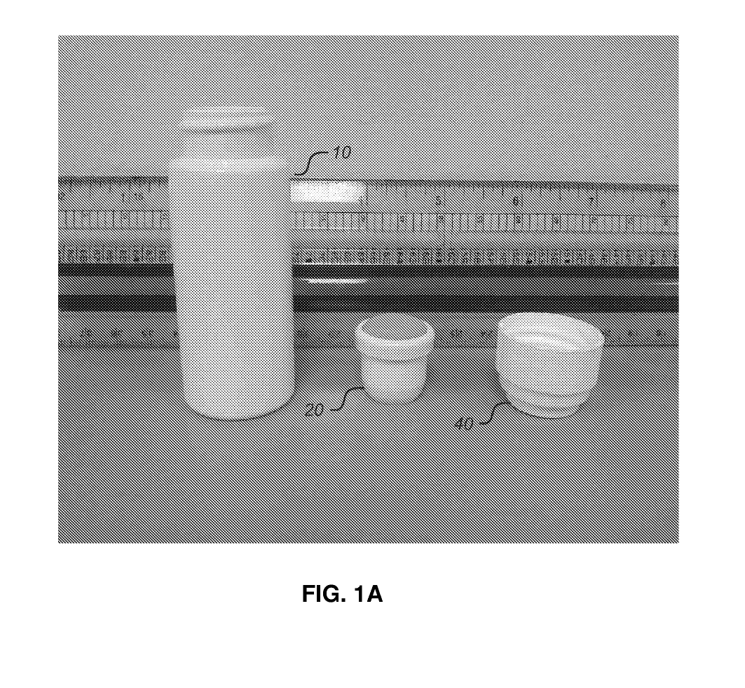

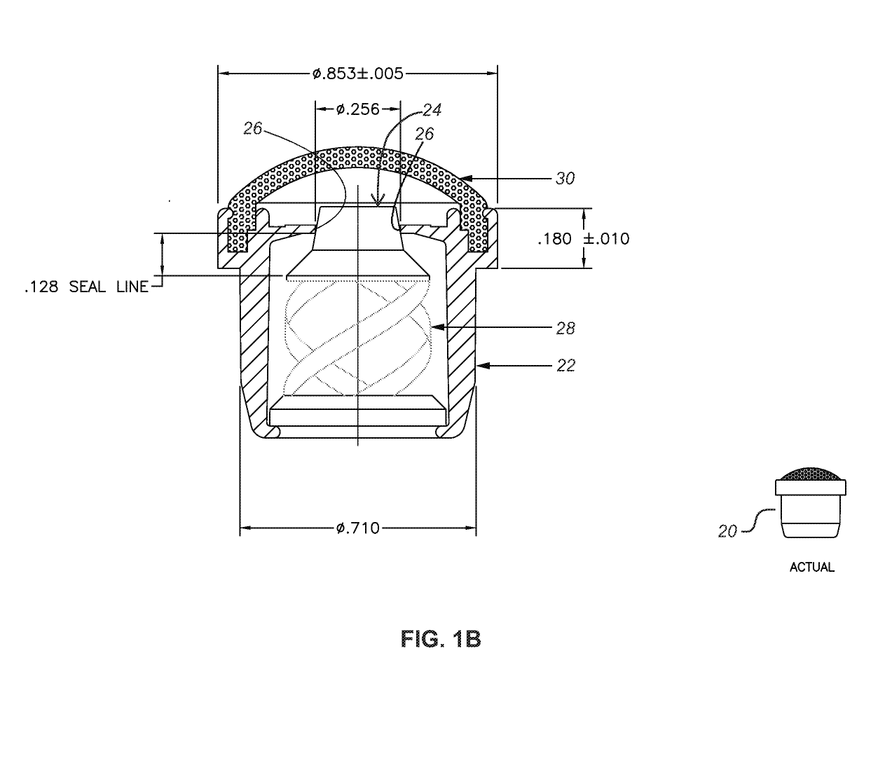

Liquid formulations containing anti-neoplastic agents, such as 5-FU, podophyllotoxin and imiquimod, for the treatment of premalignant and malignant skin lesions are filled into plastic or glass bottles 10 having a control-flow applicator 20 and a cap 40 (FIG. 1A). Referring to FIG. 1B, the applicator 20 having a housing 22 enclosing a valve plug 24 seated within an aperture 26 defined by the housing and a valve spring 28 configured to provide a force for directing the valve plug in the direction of the aperture. Attached to the housing and disposed over the aperture is a cover pad 30 constructed of a porous material such that when the bottle is inverted and the applicator is pressed against a surface, the force of the valve spring is overcome unseating the valve plug from the aperture allowing fluid to flow out from the bottle through the cover pad to the surface. These bottle applicators are then utilized to apply liquid formulations of these toxic anti-neoplastic agents without the patient having to have any hand contact with the medicament being utilized.

Liquid formulations include those suitable for topical administration, for example, solutions, suspensions, lotions, creams and gels. In such formulations, the anti-neoplastic active agents may, for example, be present as follows: 5-FU in the amount of about 0.1% to 10.0% by weight; podophyllotoxin (podofilox) in the amount of about 0.1% to 15.0% by weight; imiquimod in the amount of about 1% to 10% by weight.

Suitable pharmaceutical vehicles and methods of preparing such formulations as are within the scope of the invention will be readily apparent to and understood by those skilled in the art.

### EXAMPLES

### Example 1

A 5-fluorouracil (5-FU) was incorporated into an alcoholic solution at the concentration of 1.0% by weight. The resulting solution was bottled in plastic bottles with control-flow applicators and caps made by Dab-O-Matic Corporation, Mount Vernon, N.Y.

### Example 2

5-FU is incorporated into an aqueous suspension at a concentration of 10.0% by weight, and then bottled in a glass bottle with a control-flow applicator and cap.

### Example 3

Podophyllotoxin in a concentration of 0.5% by weight is incorporated into an inert lotion vehicle and then poured into plastic bottles with control-flow applicators and caps.

### Example 4

Podophyllotoxin is incorporated into an alcoholic gel in a concentration of 15.0%, and this gel is packaged in a glass bottle with a control-flow applicator and cap.

### Example 5

Imiquimod in concentrations ranging from 1.0% to 10.0% by weight is incorporated into a fluid cream and the resulting creams are packaged in plastic bottles with control-flow applicators and caps.

While the foregoing is a description of the preferred embodiments of the invention, it will be readily apparent to those skilled in the art that various modifications may be made therein without departing from the true scope and spirit of the invention as set forth in the appended claims.

What is claimed is:

1. A method of packaging one or more anti-neoplastic pharmaceutical agents for application to a premalignant or malignant cutaneous skin lesion, the method comprising:
(a) providing a bottle having a control-flow applicator top and a cap;
(b) filling the bottle with a liquid formulation of an anti-neoplastic agent; and
(c) securing the control-flow applicator top and cap to the bottle to permit a patient to apply the liquid formulation to the skin lesion without the patient having physical contact with the liquid formulation.

2. The method of claim 1 wherein the bottle is plastic or glass.

3. The method of claim 1 wherein the applicator top includes a housing enclosing a valve plug.

4. The method according to claim 3, wherein the valve plug is seated within an aperture defined by the housing.

5. The method of claim 4, wherein the applicator top further comprises a valve spring configured to provide a force for directing the valve plug in the direction of the aperture.

6. The method of claim 3, wherein the applicator top further comprises a cover pad constructed of porous material such that when the bottle is inverted and the cover pad is pressed against a surface, the anti-neoplastic fluid will flow out of the bottle through one or more pores of the cover pad to the surface.

7. The method of claim 1 wherein the liquid formulation is selected from the group consisting of solutions, suspensions, lotions, creams or gels.

8. The method of claim 1 wherein the anti-neoplastic agent is selected from the group consisting of 5-fluorouracil (5-FU), podophyllotoxin (podofilox) or imiquimod.

9. The method of claim 8 wherein 5-FU is present in the amount of about 0.1% to about 10.0% by weight.

10. The method of claim 8 wherein podophyllotxin is present in the amount of about 0. 1% to about 15.0% by weight.

11. The method of claim 8 wherein imiquimod is present in the amount of about 1.0% to about 10.0% by weight.

12. A package for applying an anti-neoplastic pharmaceutical agent to a premalignant or malignant cutaneous skin lesion, the package comprising:
(a) a bottle including a liquid formulation of an anti-neoplastic agent; and
(b) a control-flow applicator top and a cap configured to be secured to the bottle, wherein the control-flow applicator top and cap permit a patient to apply the liquid formulation to the skin lesion without the patient having physical contact with the liquid formulation.

13. The package of claim 12 wherein the applicator top includes a housing enclosing a valve plug.

14. The package according to claim 13, wherein the valve plug is seated within an aperture defined by the housing.

15. The package of claim 14, wherein the package further comprises a valve spring configured to provide a force for directing the valve plug in the direction of the aperture.

16. The package of claim 13, wherein the package further comprises a cover pad constructed of porous material such that when the bottle is inverted and the cover pad is pressed against a surface, the anti-neoplastic fluid will flow out of the bottle through one or more pores of the cover pad to the surface.
